SEQUENCE LISTING (1 sequence)

SEQ ID NO: 1 - protein, 153 aa, bos taurus
MKFLCVLLLASLAATSLAILNKPEDETHLEAQPTDASAQFIRNLQISNEDLSKEPSISREDLISKEQIVIRSSRQPQSQNPKLPLSILKEKHLRNATLGSEETTEHTPSDASTTEGKLMELGHKIMRNLENTVKETIKYLKSLFSHAFEVVKT

(19) United States
(12) Patent Application Publication
(10) Pub. No.: US 2017/0088813 A1
(43) Pub. Date: Mar. 30, 2017

(54) Biologically active C-terminal arginine-containing peptides

(71) Applicant: Genentech, Inc., South San Francisco, CA (US)

(72) Inventors: James Wilkins, San Francisco, CA (US); Masaru Ken Shiratori, Burlingame, CA (US); Tim Breece, San Francisco, CA (US)

(21) Appl. No.: 14/757,925

(22) Filed: Dec. 24, 2015

Related U.S. Application Data
(62) Division of application No. 12/932,207, filed on Feb. 18, 2011.
Application No. 12/932,207 is a division of application No. 12/231,917, filed on Sep. 5, 2008, now abandoned.
(60) Provisional application No. 60/967,644, filed on Sep. 5, 2007.

(51) Int. Cl. C12N 5/00; C07K 5/072; C07K 5/087; C07K 5/062; C07K 5/093; C07K 5/083

(52) U.S. Cl. CPC C12N 5/0018; C07K 5/0819; C07K 5/081; C07K 5/0808; C07K 5/0806; C07K 5/0812; C07K 5/06052; C07K 5/06104; C07K 5/06113; C12N 2501/998; C12N 2510/02

(57) ABSTRACT

The present invention concerns the separation, identification and characterization of active peptide fragments from peptones.

This is a divisional application of application Ser. No. 12/932,207, filed Feb. 18, 2011, which is a divisional application of application Ser. No. 12/231,917, filed Sep. 5, 2008 under 37 CFR 1.53(b)(1), claiming priority under 35 USC 119(e) to provisional application No. 60/967,644 filed Sep. 5, 2007, the contents of which are incorporated herein by reference.

### FIELD OF THE INVENTION

The present invention concerns the separation, identification and characterization of active peptide fragments from peptones.

### BACKGROUND OF THE INVENTION

Hydrolysates of animal and plant tissues are often added to culture media for prokaryotic and eukaryotic cells to boost viable cell number and recombinant protein product titer. Thus, peptones from diverse sources have been widely employed in cell culture medium formulations to enhance cell culture growth, longevity and productivity. However, their presence as media additives for production of therapeutic proteins presents several challenges due to their undefined nature.

The use of animal-derived components as media additives for production of therapeutic proteins by mammalian cells can cause viral, mycoplasma and prion contaminations (Kallel et al., J. Biotechnol. 95:195-204 (2002)). The effort of avoiding the use of animal-derived components in cell culture media is supported by both European and American regulatory authorities (Castle and Roberston, Dev. Biol. Stand. 99:191-196 (1999)). Animal serum has been successfully eliminated from media in the past decades and most of the media currently used is certified as protein-free (Froud, Dev. Biol. Stand. 99:157-166 (1999)). However, the switch to protein-free media often results in a loss of cell growth and productivity (Lee et al., J. Biotechnol. 69:85-93 (1999)).

As a result, protein hydrolysates (also known as peptones) are frequently used in cell culture media as nutrient additives because some of them can be considered protein-free (Burteau et al., In Vitro Cell. Dev. Biol.—Anim. 39(7):291-296 (2003)). Peptones for cell culture are typically manufactured by enzymatic digestion of a variety of biologically based starting materials: animal tissues, milk-derived products, microorganisms or plants.

The general effect of peptones is an enhancement of cell growth and an increase of productivity, with the same product quality as compared to standard medium (Jan et al., Cytotechnology 16:17-26 (1994); Heidemann et al., Cytotechnology 32:157-167 (2000); Sung et al., Appl. Microbiol. Biotechnol. 63:527-536 (2004)). Several papers have shown that peptones contain materials such as free amino acids, oligopeptides, iron salts, lipids and trace elements (Franek et al., Biotechnol. Prog. 16:688-692 (2000); Martone et al., Bioresour. Technol. 96:383-387 (2005)). One study investigated the potential roles of peptones and showed that they can have a nutritional effect (Heidemann et al., Cytotechnology 32:157-167 (2000)). Also, Schlaeger, J. Immunol. Meth. 194:191-199 (1996), demonstrated that a particular protein hydrolysate displays anti-apoptosis properties.

Despite the numerous investigations on peptone effects in cell culture, their mechanism of action is still unknown. And even though peptones have been shown to be beneficial in cell culture processes their usage also has many disadvantages. First, since some peptones are animal-derived, the similar problems are encountered as with serum usage. In addition, peptones from other sources may also be a potential source of adventitious agents. Second, the uncertainty of the “active” components in peptone and their mechanism of action makes it difficult if not impossible to have a measure of quality on peptone lots used in cell culture manufacturing processes. Finally, because source materials and processes in the manufacture of peptone are not standardized or universally interchangeable, the peptones themselves are by nature a single-sourced raw material. Therefore, there is the opportunity to better characterize peptones in order to reduce or eliminate these disadvantages.

It would, therefore, be advantageous to understand the composition and identity of the active components in peptones in order to create “defined media,” comprising well-specified active components in well-defined, reproducible concentrations. In addition the identification of active peptone components is expected to lead to a better understanding of the physiology of recombinant host cells and cell lines by opening a window to the control of cell growth and death and better control of recombinant protein expression.

Therefore, efforts have developed to characterize peptone compositions and to understand the mechanisms of their effect on cell cultures.

The present invention is based on the fractionation of an animal-derived peptone, PP3 into identifiable components and, by applying modern analytical techniques including high resolution mass spectrometry, to study the nature of these compounds. The present invention is based, at least in part, on the recognition that di- and tripeptides terminating in arginine play a role in the growth- and titer-promoting activities of PP3.

### SUMMARY OF THE INVENTION

In one aspect, the present invention concerns a peptide of the formula

(X1)ₙX2R

wherein X1 and X2 may be identical or different and independently represent any amino acid other than arginine;
n is 0 or 1; and
R stands for arginine,
wherein said peptide exhibits a peptone biological activity.

In one embodiment, the peptide is obtained by fractionation of a peptone, such as a PP3 peptone.

In another embodiment, the peptide is selected from the peptides listed in Table 1.

In another embodiment, the peptide is a tripeptide.

In yet another embodiment, the peptide is a tripeptide is selected from the peptides listed in Table 1.

Peptone biological activity can, for example, be selected from the group consisting of promotion of cell growth, promotion of cell density; promotion of viable cell count; and increase of production yield in a recombinant host cell culture.

In another aspect, the invention concerns a composition comprising a di- or tripeptide as hereinabove defined.

In one embodiment, the composition is a cell culture medium.

In a further aspect, the invention concerns a cell culture comprising a peptide as hereinabove defined.

In one embodiment, the cell culture is a culture of a recombinant host cell, where the recombinant host cell can be any eukaryotic or prokaryotic host, including, without limitation, Chinese Hamster Ovary (CHO) cells and E coli cells.

The heterologous protein produced by the host cell may, for example, be an antibody (including antibody fragments) or any therapeutic protein.

In a still further aspect, the invention concerns a mixture of two or more peptides as hereinabove defined.

In a different aspect, the invention concerns a combinatorial peptide library comprising, consisting essentially or, or consisting of, peptides of the formula

(X1)ₙX2R

wherein X1 and X2 may be identical or different and independently represent any amino acid other than arginine;
n is 0 or 1; and
R stands for arginine,
wherein at least some of said peptides exhibit a peptone biological activity.

In yet another aspect, the invention concerns a method for the recombinant production of a heterologous protein, comprising culturing a recombinant host cell containing nucleic acid encoding said protein in a culture medium under conditions suitable for the expression of said heterologous protein, wherein said culture medium comprises at least one peptide of claim 1.

These and other aspects of the invention will be apparent from the following description, including the Example.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fees.

FIG. 1 is a schematic illustration of PP3 fractionation and analysis, as described in the Example.

FIG. 2 shows the results of C18 reverse phase chromatography.

FIG. 3 shows the results of chromatography of C18 flowthrough (unbound) fraction. “Relative specific activity” is defined as the activity of an equal weight of the fraction divided by that of PP3.

FIGS. 4A and 4B show that the specific activity of the G15-purified material (fractions F3-F7) was significantly increased over that of PP3 with respect to its ability to increase both recombinant protein titer (FIG. 4A) and viable cell count (VCC; FIG. 4B).

FIG. 5. Mass spectral analysis of G-15 fraction. Top panel: MS scan of the fraction. Bottom Panels: ms/ms analysis of m/z 403.2, 389.2 and 375.2 respectively.

FIG. 6. Frequency distribution of peptides in an in silico tripeptide library terminating in R. X axis: mass of tripeptides; Y axis: frequency at each mass.

FIGS. 7A and B. Effects of PP3 and XXR peptides on VCC and product titer. A. VCC measured in the bioassay in response to addition of increasing concentrations of PP3 (Black line) or XXR peptides (Red line). B. Product Titer measured in the bioassay in response to addition of increasing concentrations of PP3 (Black line) or XXR peptides (Red line).

FIG. 8. Amino acid sequence of bovine Component-3 of proteose peptone (PP3) polypeptide (SEQ ID NO: 1).

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

The term “peptone” is used herein in the broadest sense and includes water-soluble protein derivatives obtained by partial hydrolysis of proteins, and mixtures thereof, including, without limitation, hydrolysate mixtures derived from the mucosal tissue of swine, cattle, and other animals, produced by hydrolysis with proteolytic enzymes, and well as hydrolysates from various plant tissues. The term specifically includes, without limitation, Component-3 of proteose peptone (PP3), also called lactophorin, which is a minor phosphoglycoprotein of 153 residues (Sorensen and Sorensen, J. Dairy Res. 60:535-542 (1993)) found in bovine milk (accession number in SwissProt databank: P80195; GenBank Accession No. CAA58309), and homologous proteins characterized in milk of other species as camel (Beg et al., FEBS Lett. 216:270-274 (1987), llama (Cantisani et al., J. Biochem. Biophys. Methods 21:227-236 (1990), ewe, and goat (Sorensen et al., J. Dairy Sci. 80:3176-3181(1997) and Lister et al., J. Dairy Res. 81:2111-2115 (1998). Up to now, PP3 has not been found in human milk. PP3 is available from commercial sources, such as, for example, Difco Laboratories, Detroit, Mich.

The term “peptide” is used herein to refer to a compound containing two or more amino acids in which the carboxyl group of one acid is linked to the amino group of the other by an amide or “peptide” bond. The definition specifically includes peptides formed of at least two amino acid residues, and in particular di- and tri-peptides.

By “protein” is meant a sequence of amino acids for which the chain length is sufficient to produce the higher levels of tertiary and/or quaternary structure. Typically, the protein herein will have a molecular weight of at least about 15-20 kD, preferably at least about 20 kD. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The term “biological activity” or “peptone biological activity” in relation to the peptides of the present invention is used to refer to any biological activity known to be exhibited by peptones, including, without limitation, the ability to promote cell growth and/or cell density and/or cell viability, and/or production efficiency of heterologous polypeptides in recombinant host cultures.

The term “chromatography” refers to the process by which a solute of interest in a mixture is separated from other solutes in a mixture as a result of differences in rates at which the individual solutes of the mixture migrate through a stationary medium under the influence of a moving phase, or in bind and elute processes.

The term “affinity chromatography” and “protein affinity chromatography” are used interchangeably herein and refer to a protein separation technique in which a protein of interest or antibody of interest is reversibly and specifically bound to a biospecific ligand. Preferably, the biospecific ligand is covalently attached to a chromatographic solid phase material and is accessible to the protein of interest in solution as the solution contacts the chromatographic solid phase material. The protein of interest (e.g., antibody, enzyme, or receptor protein) retains its specific binding affinity for the biospecific ligand (antigen, substrate, cofactor, or hormone, for example) during the chromatographic steps, while other solutes and/or proteins in the mixture do not bind appreciably or specifically to the ligand. Binding of the protein of interest to the immobilized ligand allows contaminating proteins or protein impurities to be passed through the chromatographic medium while the protein of interest remains specifically bound to the immobilized ligand on the solid phase material. The specifically bound protein of interest is then removed in active form from the immobilized ligand with low pH, high pH, high salt, competing ligand, and the like, and passed through the chromatographic column with the elution buffer, free of the contaminating proteins or protein impurities that were earlier allowed to pass through the column. Any component can be used as a ligand for purifying its respective specific binding protein, e.g. antibody.

The terms “non-affinity chromatography” and “non-affinity purification” refer to a purification process in which affinity chromatography is not utilized. Non-affinity chromatography includes chromatographic techniques that rely on non-specific interactions between a molecule of interest (such as a protein, e.g. antibody) and a solid phase matrix.

A “cation exchange resin” refers to a solid phase which is negatively charged, and which thus has free cations for exchange with cations in an aqueous solution passed over or through the solid phase. A negatively charged ligand attached to the solid phase to form the cation exchange resin may, e.g., be a carboxylate or sulfonate. Commercially available cation exchange resins include carboxy-methyl-cellulose, sulphopropyl (SP) immobilized on agarose (e.g. SP-SEPHAROSE FAST FLOW™ or SP-SEPHAROSE HIGH PERFORMANCE™, from Pharmacia) and sulphonyl immobilized on agarose (e.g. S-SEPHAROSE FAST FLOW™ from Pharmacia). A “mixed mode ion exchange resin” refers to a solid phase which is covalently modified with cationic, anionic, and hydrophobic moieties. A commercially available mixed mode ion exchange resin is BAKERBOND ABX™ (J. T. Baker, Phillipsburg, N.J.) containing weak cation exchange groups, a low concentration of anion exchange groups, and hydrophobic ligands attached to a silica gel solid phase support matrix.

The term “anion exchange resin” is used herein to refer to a solid phase which is positively charged, e.g. having one or more positively charged ligands, such as quaternary amino groups, attached thereto. Commercially available anion exchange resins include DEAE cellulose, QAE SEPHADEX™ and FAST Q SEPHAROSE™ (Pharmacia).

### B. Detailed Description

Fractionation and Analysis of Peptones

The present invention is based on the identification of active peptone fragments, using a variety of chromatography and other separation techniques.

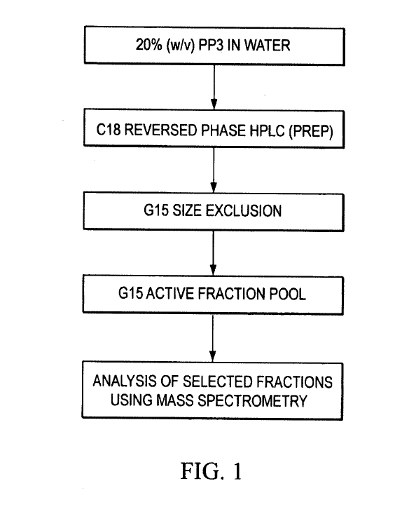

Fractionation of peptones can be performed by techniques well known in the art, including a combination of chromatographic steps. A typical scheme for the fractionation and analysis of the peptone PP3 is illustrated in FIG. 1. The first step in this scheme is reversed phase HPLC (RP HPLC), which serves to separate components based on their hydrophobic character. Compounds bind to reversed phase HPLC columns in high aqueous mobile phase and are eluted from RP HPLC columns with high organic mobile phase. Thus, peptides can be separated using this technique by running linear or nonlinear gradients of an appropriate organic solvent.

The flow-through fraction from the reversed phase HPLC can then be subjected to further chromatographic separation steps, such as size-exclusion chromatography, as shown in FIG. 1. Size exclusion chromatography (SEC) relies on sorting of molecules of different hydrodynamic radius based on the time these molecules spend within a chromatographic column. Partitioning occurs as a result of the molecules spending more or less time within the volume of the stationary phase which makes up the column. Molecules with large hydrodynamic radius elute early during the separation; molecules with smaller radii elute later. SEC resins are well known in the art and are commercially available from a variety of manufacturers. In the experiments described in the Example below, SEPHADEX™ G15 size exclusion column (Amersham Biosciences) was used, but other size exclusion columns are also suitable.

If desired, peptones can be sub-fractionated by mixed-mode chromatography. In this technique, the stationary phase contains two distinct binding domains in a single chromatographic ligand. The separation principles commonly coupled are ion-exchange and RP chromatography. The application of mixed-mode chromatography is generally required when reverse phase chromatography (RP chromatography) fails to separate structurally close peptides. Currently, several manufacturers offer mixed-mode resins commercially that show different specificities, including the Hypercarb resin (Thermo Scientific), Oasis® HLB resin (Waters Corporation) and the Dowex Optipore® SD-2 resin (Dow Chemical Company). The Oasis sorbent is a copolymer of divinylbenzene and N-vinyl pyrrolidone. The hydrophilic-lipophilic-balanced composition allows for strong RP retention and improved wetting of the pores relative to traditional RP resins. Therefore, both polar and non-polar compounds can be adsorbed. The average size of the Oasis beads is 30 μm. The composition of the Optipore resin is similar to that of the Oasis resin (divinylbenzene with tertiary amines) giving it properties of both RP and a weak anion-exchange absorber. The particles average size is a few hundred μm.

At each purification step, the fractions obtained can be analyzed for biological activity and/or subjected to various analytical methods in order to determine their composition.

Assays of Peptide Activity

The peptone fractions generated can be tested using known bioassays, including a cell based bioassay described in the Example. Assays of the biological activity of peptone-derived peptides are typically based on the measurement of various culture parameters in cell cultures including the peptide in question in the culture medium. Such parameters include, for example, cell density, viable cell count, productivity of a heterologous polypeptide (e.g. antibody) produced, etc. Such assays are disclosed, for example, in Franek et al., Biotechnol. Prog. 16:688-692 (2000); Franek et al., Biotechnol. Prog. 18:155-158 (2002); Franek et al., Biotechnol. Prog. 19:169-174 (2003); Franek et al., Biotechnol. Prog. 21:96-08 (2005).

Determination of the Composition of Peptone Fractions

Thus, the compositions of peptones and peptone fractions can be analyzed by mass spectrometry (MS), NMR, ICPMS, amino acid analysis, and various combinations of these and other techniques well known in the art.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionisation (ESI) methods. There are several versions of mass analyzer, including, for example, MALDI-TOF and triple or quadrupole-TOF, or ion trap mass analyzer coupled to ESI. Thus, for example, a Q-Tof-2 mass spectrometer utilizes an orthogonal time-of-flight analyzer that allows the simultaneous detection of ions across the full mass spectrum range. For further details see, e.g. Chemusevich et al., J. Mass Spectrom. 36:849-865 (2001).

If desired, the amino acid sequences of the peptide fragments and eventually the proteins from which they derived can be determined by techniques known in the art, such as certain variations of mass spectrometry, or Edman degradation.

While the present invention is illustrated by peptide fragments identified in the degradation products of a particular peptone, it is expected that other peptones will have peptide components with similar structural and functional characteristics.

Combinatorial Peptide Libraries

Combinatorial peptide libraries are well known in the art, and methods for the generation and screening of such libraries are described in standard textbooks, such as, for example, in Combinatorial Peptide Library Protocols (Methods in Molecular Biology), Shmuel Cabilly, ed., Humana Press Inc. 1998.

Combinatorial peptide libraries may be in different formats. For example, the so called “one bead one compound” libraries contain thousands of beads, each bearing multiple copies of a single library compound (Lam et al., Nature, 354, 82-84 (1991)). The library is screened for a desired activity and any active bead(s) is/are isolated. The active compound attached to the identified bead(s) identified is then characterized by conventional methods, such as, for example, by Edman degradation. Alternatively, a reductive approach may be employed. In another method, Houghten's positional fixing methodology (Bioorg. Med. Chem. Lett., 14, 1947-1951 (2004)), a set of related libraries is generated before screening. Each library contains compounds with one specific residue fixed as a single building block and the remaining residues fully randomized. Different libraries have a different building block at the fixed position. Screening this set of libraries, for a desired activity, enables direct identification of the optimum building block at the fixed residue. This process may be carried out sequentially, optimizing one residue at a time, or alternatively all of the sets of libraries may be screened simultaneously to allow the optimum library compound to be identified directly from a single round of screening.

Using the peptides specifically disclosed herein, the combinatorial peptide libraries can be used to identify additional peptides with improved chemical and/or biological characteristics.

Peptide Synthesis

The peptides identified in accordance with the present invention can be synthesized by conventional methods of peptide synthesis, such as, for example, solid-phase synthesis methods, which can be performed in a variety of forms, such as, for example, using Fmoc (9H-fluoren-9-yl-methoxy-carbonyl) or Boc (tert-butoxycarbonyl) protecting groups to protect the N-termini of amino acid monomers used in the synthesis. Automated synthesizers are commercially available for both techniques, but solid phase peptide synthesis can also be performed manually. For further details see, for example, Atherton, E., Sheppard, R. C. (1989). Solid Phase peptide synthesis: a practical approach. Ocford, England: IRL Press. ISBN 0199630674; and Stewart, J. M., Young, J. D. (1984). Solid phase peptide synthesis, 2^{nd} edition, Rockford: Pierce Chemical Company, 91. ISBN 0935940030.

Uses of the Di- and Tri-Peptides

The di- and tri-peptides of the present invention can be used as components of growth media used for the production of recombinant polypeptides, including antibodies.

Recombinant polypeptides, such as antibodies, can be produced in a variety of eukaryotic and prokaryotic host organisms.

Suitable host cells for the expression of glycosylated polypeptides, such as antibodies, can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed bySV40 (COS-7, ATCC CRL 1651); human embryonic kidney line (293 or 293 cells subloned for growth in suspension culture, Graham et al, J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

For example, recombinant polypeptides, such as antibodies, can be produced in dp12.CHO cells, the production of which from CHO-K1 DUX-B11 cells as described in EP307247. CHO-K1 DUX-B 11 cells were, in turn, obtained from CHO-K1 (ATCC No. CCL61 CHO-K1) cells, following the methods described in Simonsen, C. C., and Levinson, A. D., (1983) Proc. Natl. Acad. Sci. USA 80:2495-2499 and Urlaub G., and Chasin, L., (1980) Proc. Natl. Acad. Sci USA 77:4216-4220. In addition, other CHO-K1 (dhfr) cell lines are known and can be used.

The mammalian host cells used to produce peptides, polypeptides and proteins can be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM, Sigma) are suitable for culturing the host cells. In addition, any of the media described in Ham and Wallace (1979), Meth. in Enz. 58:44, Barnes and Sato (1980), Anal. Biochem. 102:255, U.S. Pat. Nos. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The peptides of the present invention can be used, individually or in various combinations, as components of any commercially available or custom made culture medium.

The use of the peptides herein is not limited to the culturing of mammalian, or, in general, eukaryotic host cells. The peptides of the present invention also find utility in cell cultures of prokaryotic host organisms. Exemplary prokaryotic host cells include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E coli W3110 (ATCC 27,325) are suitable.

Further details of the present invention are provided in the following non-limiting Example.

### Example

### Molecular Identification of Biologically Active C-Terminal Arginine Containing Tripeptides Using a Combinatorial Library

### Materials and Methods

TubeSpin Bioassay

The TubeSpin bioassay was designed to examine the effects of a cell culture medium additive on cellular proliferation and productivity (as determined by viable cell density and concentration of antibody produced, respectively) in a 5 milliliter (mL) working volume using an initial viable cell density of 1.5×10̂6 cells/mL.

Each culture was prepared in a volume of 7.15 mL from which 5 mL are transferred to the assay tube. The assay tube is a 50 mL centrifuge tube with a specialized cap that contains a membrane with a 0.2 micron pore-size to facilitate the flow of gases. The 2.15 mL that remained was used to measure viable cell density (to confirm that both the target density has been achieved and that the cells are viable), osmolality and metabolic data (pH, glutamine, glutamate, glucose, lactate, ammonium, sodium, potassium).

Depending upon the starting concentration and osmolality of the desired medium additive, a target concentration for that additive was determined. From this data, the volume of the additive required, the volume of sodium chloride required to achieve physiological osmolality in the final culture, and the volume of water necessary to achieve 7.15 mL are calculated. Using these values a preparative solution of medium additive, sodum chloride, and water was assembled.

Cells in Selective Genentech Essential Medium (GEM) were then medium-exchanged into a production-quality medium referred to as GEM2. Cells in GEM2 were then added to each preparative tube to achieve the stated 1.5×10̂6 cells/mL in 7.15 mL. Each assay tube then received 5 mL and was incubated at 37 degrees centigrade for 4.75 days.

At the end of the incubation period the viable cell density, osmolality, and metabolic profile were once again measured, and a sample of the assay culture was sent to a separate antibody quantification assay to determine its concentration.

C18 Reversed Phase Preparative Chromatography

A 20% (w/v) aqueous solution of PP3 was prepared and was used immediately. A 5×5 cm stainless steel column was packed with Waters Preparative C18 silica (125 Å pore size; 55-105 micron particle size). The column was preequilibrated with 10 volumes of 0.02% (w/v) trifluoroacetic acid (TFA) in water (Buffer A) and was operated at a flowrate of 60 ml/min. 200 mL of the above PP3 solution was loaded onto the column. Solvent control and effluent monitoring of the column was achieved using an Akta chromatography system controlled by Unicorn software (GE Healthcare). Following the load, the column was washed with buffer A until the absorbance of the effluent at 280 nm reached baseline. The column was then washed with 85% (v/v) acetonitrile (ACN) containing 0.02% TFA to elute bound material. Fractions were assayed for biological activity using the bioassay

G15 Sephadex Preparative Chromatography

The flowthrough material from the C18 was concentrated by rotary evaporation and was further fractionated on a G-15 Sephadex column (7 cm dia.×50 cm l.). The column was equilibrated with deionized water prior to loading and the entire C18 flowthrough pool was loaded. Fractions were collected and assayed for activity using the bioassay described above.

Hypercarb Analytical Chromatography

Samples of active fractions were analyzed using a Hypercarb (graphite) column (5 micron particle size; column dimensions: 0.1 mm×50 mm). The buffers used were Buffer A: aqueous 0.02% TFA; and Buffer B: 85% ACN+0.02% TFA. Various volumes (0-10 microliters) were loaded onto the column which was preequilibrated with Buffer A and eluted with a linear gradient going to 50% Buffer B over 25 minutes. The column was operated at a flowrate of 0.1 ml/min. Solvent control was achieved with an Agilent 1100 HPLC equipped with diode array detector and an MSD single quadrapole mass spectrometer. The entire system was under the control of Agilent Chemstation software.

Mass Spectrometry

High resolution mass spectrometry was performed using a Q-TOF (Waters Corporation) or an Orbitrap mass spectrometer (Thermofinnigan). The instruments were operated in the static spray mode and mass accuracy was found to be <5 ppm in both cases. Spectra were used to obtain accurate mass information which was used to correlate with peptide library information.

Bioinformatics

In silico libraries of dipeptides and tripeptides were constructed with the necessary mass accuracies to correlate with masses determined as discussed above.

Peptide Synthesis

A tripeptide library was created using Fmoc solid phase peptide synthesis. The library was designed to contain all possible tripeptides terminating in arginine (R), i.e. XXR.

### Results

Fractionation of PP3

The scheme for PP3 fractionation and analysis is shown diagrammatically in FIG. 1.

C18 Reversed Phase HPLC

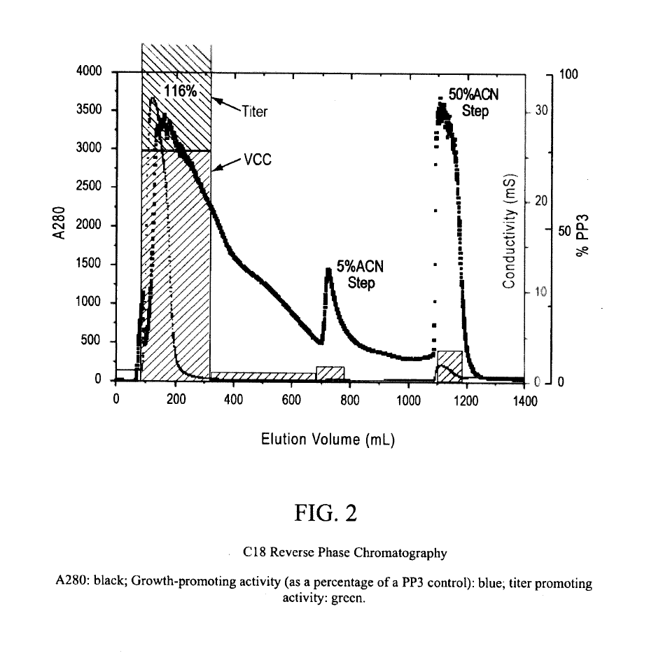

The initial fractionation of PP3 was accomplished using reversed phase HPLC. The chromatogram shown in FIG. 2 is representative of the reversed phase chromatography profile of PP3. Most of the mass (˜70% of the initial dry weight) flows through the column as does most of the biological activity.

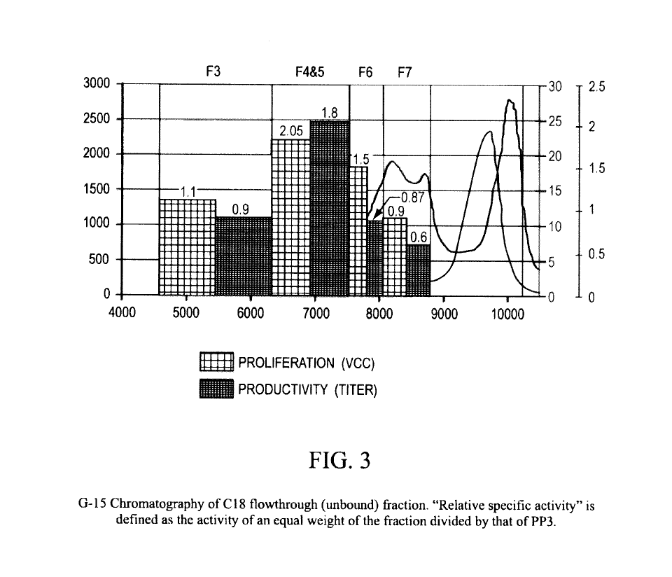

The flowthrough fraction from C18 was concentrated and loaded onto G-15 Sephadex for size exclusion chromatography, the results of which are shown in FIG. 3.

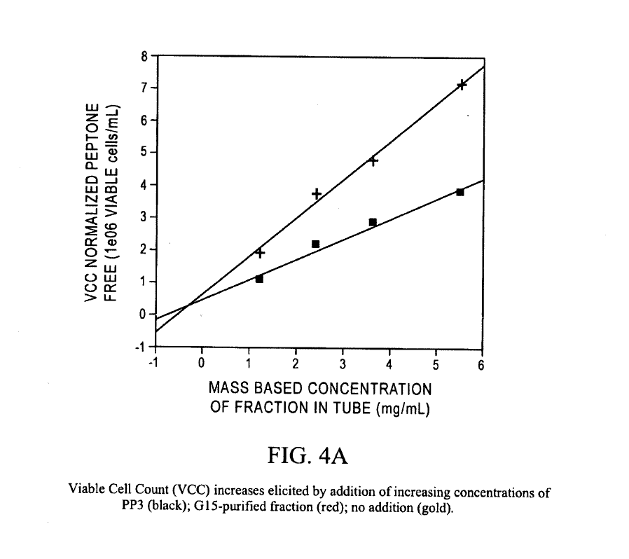

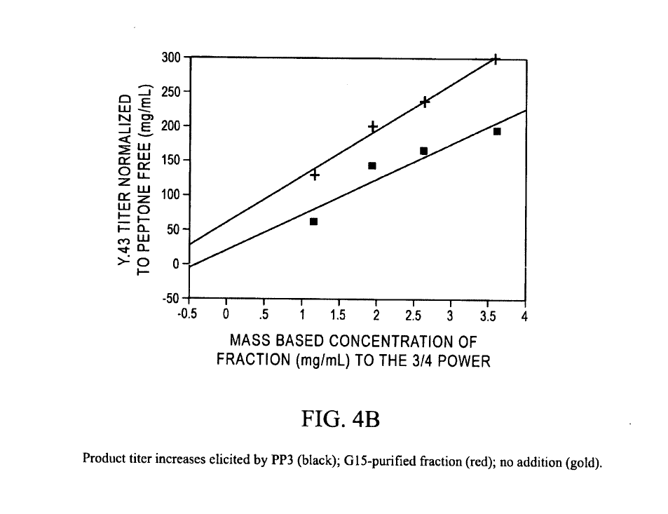

Fractions from the G15 column were analyzed for bioactivity. The specific activity of the G15-purified material (fractions F3-F7) was significantly increased over that of PP3 with respect to its ability to increase both recombinant protein titer (FIG. 4A) and viable cell count (VCC; FIG. 4B).

Mass Spectrometry on G15 Fraction Pool

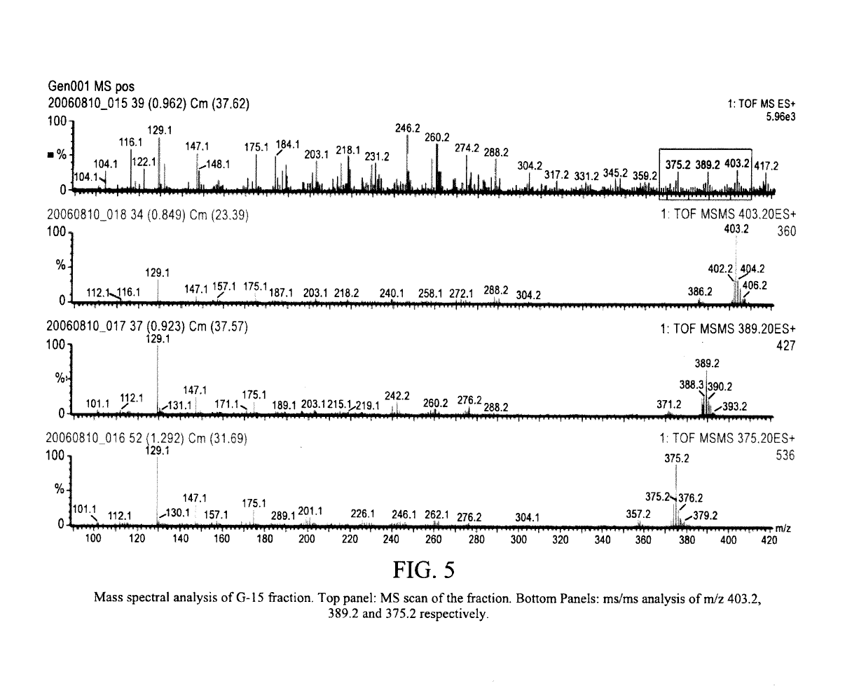

Active fractions were analyzed using mass spectrometry in order to obtain information about the molecular nature of the active components. FIG. 5 shows a representative mass spectrum obtained from a sample of G15 pool obtained as described above. The spectrum contains numerous peaks with masses <500 Da. One striking feature of the spectra was the fact that they contained many peaks differing in mass by 14 mass units in a repeating fashion. Also, a peak at 175.1 m/z (FIG. 5) was identified as arginine by exact mass and subsequent elemental composition analysis. Further, analysis of three of the masses (403.2, 389.2 and 375.2 m/z; marked by the box) from the spectrum shown in FIG. 5 using collision induced dissociation (CID) revealed similar fragmentation patterns in the mass range <200 Da. This included the identification of an intact arginine residue at 175.1 m/z from each of the three masses. In addition, based on exact mass measurements, these molecules were identified as peptides with the following possible identities:

### 403.2 Da: EVR; DIR; DLR

### 389.2 Da: LTR; ITR

### 375.2 Da: SIR; SLR; TVR

The order of the first 2 amino acids in the above sequences could also be reversed. Several possibilities exist at each mass because of the overlap in combinations of individual amino acids.

Creation of an in Silico Library of Tripeptides Containing Arginine

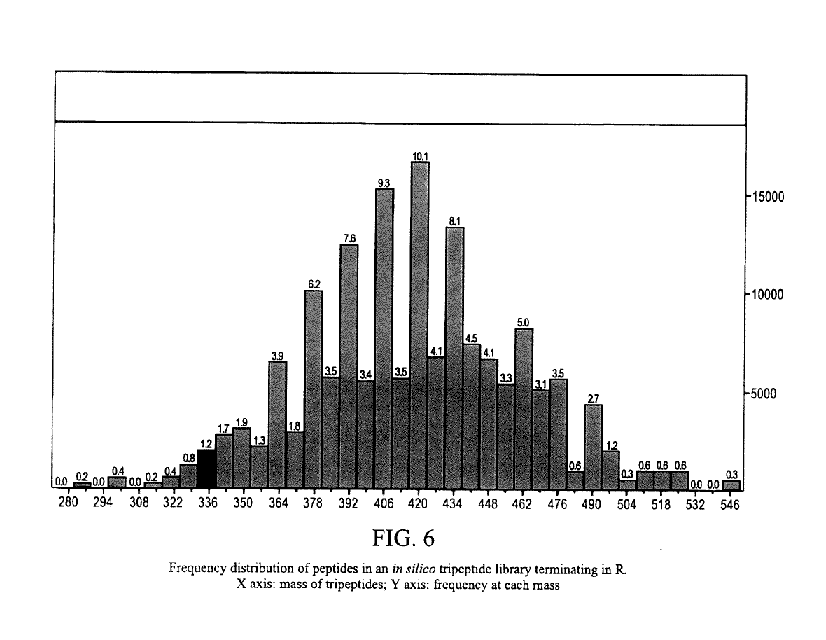

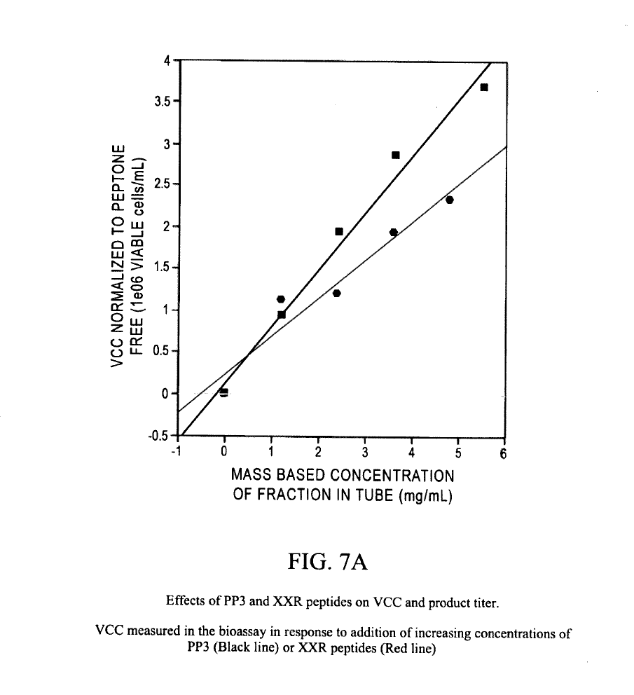

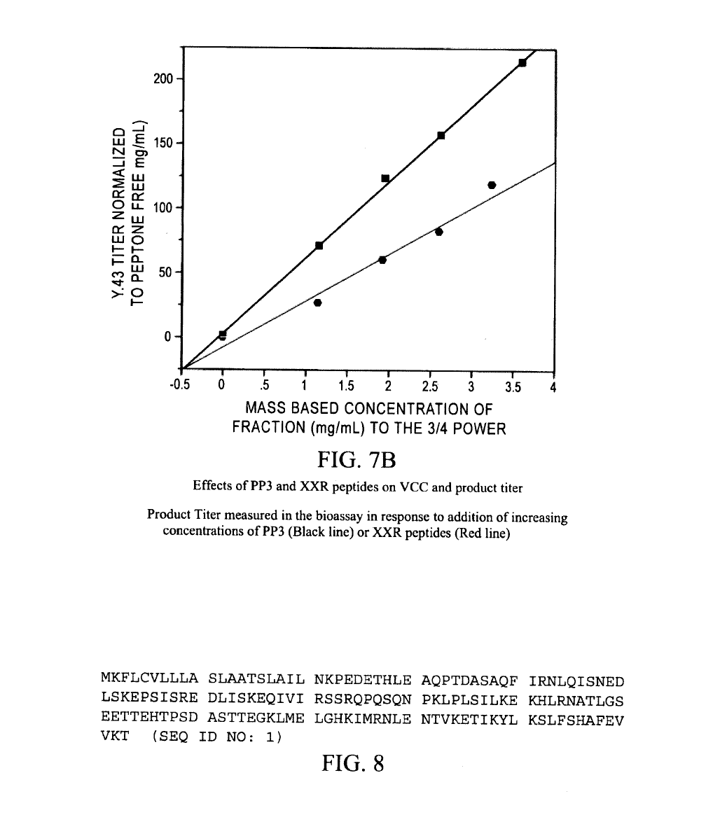

The 14 mass-unit repeating structure seen in mass spectra from peptone fractions and the presence of C-terminal arginine in a group of these molecules suggested the presence of an enzymatically-created “combinatorial library” of peptides in PP3 arising from the animal tissues. To further investigate this idea, we decided to create an in silico library of C-terminal arginine-containing tripeptides. The library contains 400 possible peptides and its mass composition and distribution is represented in the graph shown in FIG. 6. The distribution indeed resembles the 14 mass-unit differences seen in the peptone fractions with repeating peaks at intervals of 14 Da.

Construction and Analysis of a Real XXR Combinatorial Peptide Library

In order to further explore the idea that C-terminal arginine containing peptides might be responsible for some of the activity seen in PP3 fractions, we made a combinatorial library of these molecules using solid phase (Fmoc) peptide synthesis. The library containing lyophilized peptides was dissolved in deionized water and the pH of the solution was adjusted to ˜7.0 with NaOH. The solution was then desalted using a G-15 column. A fraction of the library was collected, concentrated by rotary evaporation and assayed by mass spectrometry and by bioassay. Mass spectral analysis revealed the presence of both di- and tripeptides. Dipeptides found in the mixture were thought to arise from a certain proportion of failed peptide synthesis reactions.

Mass spectrometry revealed the presence of numerous peaks, which were compared with the in silico library referred to above. Table 1 lists the masses, identities and relative intensities of the peptides found in this fraction. The assignments are based on accurate mass measurements with errors <5 ppm. This list represents a partial list of all the peptides present in this fraction.

Bioassay of G-15 XXR Fraction

The XXR peptides prepared as noted above were assayed for activity in the bioassay and found to have significant positive effects on both cell growth and titer.

### Summary

The results presented in this example show that PP3 contains active molecules with molecular masses <500 Da. Some of these molecules were identified as C-terminal arginine containing tripeptides. A combinatorial library of these peptides showed biological activity in a 6-day bioassay. We concluded that C-terminal arginine containing tripeptides are responsible, at least in part, for the growth- and titer-promoting activities of PP3.

The invention illustratively described herein can suitably be practiced in the absence of any element or elements, limitation or limitations that is not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalent of the invention shown or portion thereof, but it is recognized that various modifications are possible within the scope of the invention claimed. Thus, it should be understood that although the present invention has been specifically disclosed by preferred embodiments and optional features, modifications and variations of the inventions embodied herein disclosed can be readily made by those skilled in the art, and that such modifications and variations are considered to be within the scope of the inventions disclosed herein. The inventions have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless or whether or not the material to be removed was specifically recited. In addition, where features or aspects of an invention are described in terms of the Markush group, those schooled in the art will recognize that the invention is also thereby described in terms of any individual member or subgroup of members of the Markush group. The steps depicted and/or used in methods herein may be performed in a different order than as depicted and/or stated. The steps are merely exemplary of the order these steps may occur. The steps may occur in any order that is desired such that it still performs the goals of the claimed invention.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims.

All U.S. patents and applications; foreign patents and applications; scientific articles; books; and publications mentioned herein are hereby incorporated by reference in their entirety as if each individual patent or publication was specifically and individually indicated to be incorporated by reference, including any drawings, figures and tables, as though set forth in full.

1-23. (canceled)

24. A culture medium comprising an isolated C-terminal arginine-containing di- or tri-peptide selected from:
a) the tripeptides EVR, DPR, TVR, EIR, ELR, INR, LNR, QVR, AVR, GIR, GLR, IVR, LVR, ITR, LTR, DTR, ESR, GGR, DVR, ILR, LLR, AIR, ALR, ADR, AGR, DQR, ENR, DMR, EMR, GTR, FPR, LMR, IPR, and LPR;
b) the tripeptides EVR, VER, LTR, TLR, ITR, TIR, TVR, and VTR; and
c) the dipeptides DR, VR, ER, NR, and QR, wherein said peptide exhibits a peptone biological activity.

25. The culture medium of claim 24, wherein said peptide is obtained by fractionation of a peptone or is chemically synthesized.

26. The culture medium of claim 24 wherein said peptide is selected from the peptides EVR, DPR, TVR, EIR, ELR, INR, LNR, QVR, AVR, GIR, GLR, IVR, LVR, ITR, LTR, DTR, ESR, GGR, DVR, ILR, LLR, AIR, ALR, ADR, AGR, DQR, ENR, DMR, EMR, GTR, FPR, LMR, IPR, LPR, DR, VR, ER, NR, and QR.

27. The culture medium of claim 24 wherein said peptide is a tripeptide.

28. The culture medium of claim 27 wherein said tripeptide is selected from the peptides EVR, DPR, TVR, EIR, ELR, INR, LNR, QVR, AVR, GIR, GLR, IVR, LVR, ITR, LTR, DTR, ESR, GGR, DVR, ILR, LLR, AIR, ALR, ADR, AGR, DQR, ENR, DMR, EMR, GTR, FPR, LMR, IPR, and LPR.

29. The culture medium of claim 24 wherein said peptone biological activity is selected from the group consisting of promotion of cell growth, promotion of cell density; promotion of viable cell count; and increase of production yield in said recombinant host cell culture.

30. The culture medium of any one of claims 24 to 29, further comprising an additive selected from the group consisting of hormones, growth factors, salts, buffers, nucleosides, antibiotics, trace elements, and glucose.
